(19) United States
(12) Patent Application Publication
(10) Pub. No.: US 2021/0064897 A1
(43) Pub. Date: Mar. 4, 2021

(54) BIOMETRIC MONITORING AND PROTECTIVE DEVICE

(71) Applicant: NEW YORK UNIVERSITY, New York, NY (US)

(72) Inventor: Robert J. Cerfolio, New York, NY (US)

(21) Appl. No.: 17/012,157

(22) Filed: Sep. 4, 2020

Related U.S. Application Data
(60) Provisional application No. 62/895,659, filed on Sep. 4, 2019.

(51) Int. Cl. G06K 9/00; G06K 9/62

(52) U.S. Cl. CPC G06K 9/00892; G06K 9/00919; G06K 9/6289

(57) ABSTRACT

The present invention relates to biometric monitoring and protective devices comprising one or more sensors configured to sense and measure one or more physiological phenomena continuously or at regular intervals. The devices can be worn on the head to maintain the user's body temperature and protect sensitive areas such as the eyes and ears.

### CROSS-REFERENCE TO RELATED APPLICATIONS

This application claims priority to U.S. Provisional Patent Application Ser. No. 62/895,659, filed Sep. 4, 2019, the contents of which are each incorporated by reference herein in their entirety.

### BACKGROUND OF THE INVENTION

Hypothermia is a condition of subnormal body temperature which presents serious consequences to the patient suffering therefrom. It has been shown that nearly seventy-five percent of all patients who undergo surgical procedures develop hypothermia. This equates to approximately fourteen million patients a year in the United States alone. The hypothermic condition is brought on by many factors including anesthesia, the air conditioning of the operating room, and the infusion of cold blood, I-V solutions, or irrigating fluids.

A significant quantity of heat is lost from the head. Several methods and products have been developed to help prevent hypothermia from occurring, such as the use of infrared lamps, cotton blankets, and warm water mattresses. However, none of these methods and products are effective in preventing heat loss from the head.

Thus, there is a need in the art for improvements in temperature monitoring and maintenance of a patient. The present invention meets this need.

### SUMMARY OF THE INVENTION

In one aspect, the present invention relates to a device for monitoring and detecting biological and environmental data, comprising: a hat having an outer surface and an inner surface; one or more sensors; one or more control units operatively connected to the one or more sensors; an alerting unit operatively connected to the one or more control units; and at least one instrument holder attached to the outer surface.

In one embodiment, the alerting unit comprises one or more speakers, alarms, LEDs, vibration motors and screens. In one embodiment, the device further comprises a memory unit operatively connected to the one or more control unit, wherein the memory unit stores a set of predetermined acceptable biometric ranges. In one embodiment, the device further comprises a presence sensor operatively connected to the one or more control units, wherein one or more control units are configured to receive data from the presence sensor indicating that the device is being worn.

In one embodiment, one or more control units are configured to receive data from the presence sensor to detect that the sensors are positioned relative to the body. In one embodiment, the one or more sensors are selected from the group consisting of: a temperature sensor, a global positioning sensor, an accelerometer sensor, a gyroscope sensor, a magnetic sensor, a distance sensor, a pressure sensor, a light sensor, a biometric sensor, a blood pressure sensor, a blood glucose sensor, an oximeter sensor, and a breath sensor. In one embodiment, one or more sensors sense and measure biological and environmental data as parameters continuously or at regular intervals. In one embodiment, the one or more control units are configured to receive the parameters from the one or more sensors and to signal the alerting unit once the received parameters are outside a predetermined range.

In one embodiment, the device further comprises one or more flaps or extensions configured to cover a user's ears, forehead, eyes, or neck when worn. In one embodiment, the at least one instrument holder is adapted to hold an endotracheal tube.

In another aspect, the present invention relates to a method of monitoring biological and environmental data by wearing a device, the method comprising: providing a device having one or more sensors, one or more control units, and an alerting unit; wherein the one or more sensors are configured to sense and measure biological and environmental data as parameters continuously or at regular intervals; wherein the one or more control units are operatively connected to the one or more sensors; and wherein the alerting unit is operatively connected to the one or more control units and is configured to provide one or more alerts based on the signals received from the control unit; such that the one or more control units are configured to signal the alerting unit once the parameters received from the one or more sensors are outside a predetermined range.

In one embodiment, the alerting unit comprises one or more speakers, alarms, LEDs, vibration motors, and screens. In one embodiment, the device further comprises a memory unit configured to store the predetermined range retrievable by the one or more control units. In one embodiment, the device further comprises a presence sensor operatively connected to the control unit, wherein the control unit is configured to receive data from the presence sensor indicating that the device is being worn. In one embodiment, the one or more sensors are selected from the group consisting of: a temperature sensor, a global positioning sensor, an accelerometer sensor, a gyroscope sensor, a magnetic sensor, a distance sensor, a pressure sensor, a light sensor, a biometric sensor, a blood pressure sensor, a blood glucose sensor, an oximeter sensor, and a breath sensor.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a schematic representation of an exemplary biometric monitoring and protective device.

FIG. 2 depicts a schematic representation of an exemplary sensor module.

### DETAILED DESCRIPTION

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### The Biometric Monitoring and Protective Device

The present invention relates to a device for noninvasive determination of a user's biometrics, including body temperature, oxygen saturation levels, heart rate, etc. and environmental phenomena. In certain aspects, the device determines and monitors the biometric and environmental data continuously or at regular intervals. In one embodiment, the device is configured to be worn on the head of a user.

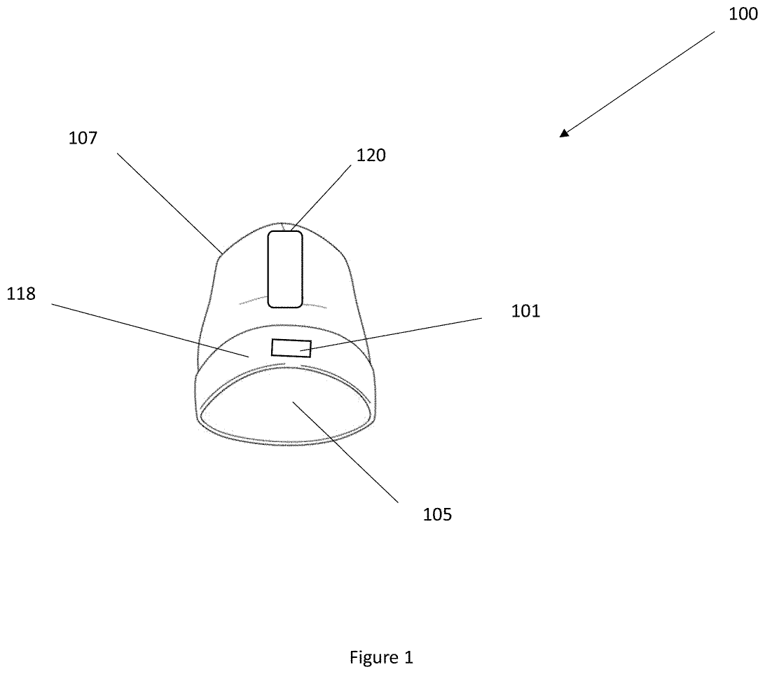

Referring now to FIG. 1, device 100 comprises a substantially dome shaped hat adapted to cover at least a part of the head of a user. Device 100 has an inner surface 105 and an outer surface 107. In one embodiment, inner surface 105 can include an elastic band or size adjustable strap. In one embodiment, inner surface 105 can include at least one padding layer 118. Device 100 can further include one or more sensor modules 101. In one embodiment, the one or more sensor modules 101 may be coupled to device 100 by an attachment means positioned between inner surface 105 and padding layer 118. The attachment means can be any means readily known in the art, including, but not limited to, an adhesive, Velcro, snap fits, buttons, and zippers.

Padding layer 118 may be manufactured from foam or other suitable material to protect the one or more sensor modules 101 from shock, moisture, and other environmental conditions that might disturb the one or more sensor modules 101 and/or compromise biological data gathering. Padding layer 118 may be coupled to a contact layer (not shown). A contact layer may be configured to directly contact or remain proximal to the skin portion of the body. The contact layer may comprise a layer of material, which may be formed as a thin sheet and manufactured from a flexible plastic or metallic material, such as thermoplastic urethane (TPU), polyvinylchloride or other known suitable plastic, metallic or other material, which conducts heat and electricity. The contact layer may be glued (or otherwise bonded) to one side of padding layer 118.

Form, size and shape of device 100 may also be adapted to cover the entire head area of the user. For example, in various embodiments, device 100 can include a flap or extension to cover a user's ears, forehead, eyes, neck, and combinations thereof. Device 100 can be made from any suitable material, including but not limited to cotton, wool, spandex, and the like. In one embodiment, device 100 is made from a stretchable fabric which has been cut and sewn to conform to the shape of a user's head. The stretch (elasticity) in the fabric allows device 100 to fit a variety of head shapes and sizes.

In certain aspects, device 100 is used in surgical settings to protect a user during an operation, such as to prevent the user's earlobes from inadvertent bending, to prevent liquid from entering the ear canal, or to protect a user's hair from water or blood. In one embodiment, device 100 is used to protect a user's eyes during surgery. For example, in one embodiment, a protective eye shield is attached to device 100, the eye shield comprising a flexible face mask, foamed plastic and soft material for lying across the user's eyes. In one exemplary embodiment, the eye shield is covered by transparent eye covers.

Device 100 can also be used to minimize heat loss from the head of the user. In one embodiment, device 100 may comprise a thermoregulatory system for cooling or heating. The system can regulate the temperature based on the signals from control unit 110. The thermoregulatory system can be any system known in the art, including an activatable thermal regulatory medium, a thermally conductive pad in conductive association with a thermoregulatory unit, and the like.

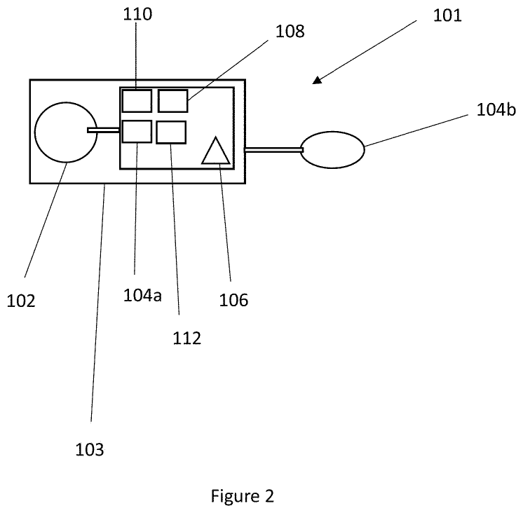

Referring now to FIG. 2, an exemplary sensor module 101 is depicted. In one embodiment, sensor module 101 comprises an outer shell 103, a battery 102, one or more sensors 104 (such as 104a and 104b), an alerting unit 106, a wireless transceiver 108, a control unit 110, a memory 112, and an instrument holding/alignment unit 120. In one embodiment, outer shell 103 is soft, flexible, and stretchable to provide optimum wear comfort when placed on a user. In one embodiment, outer shell 103 houses various components. In one embodiment, the sensor module 101 comprises various sensor slots to easily add or remove sensors based on the user's need. In one embodiment, one or more sensors 104a may be housed in a sensor module 101. In one embodiment, one or more sensors 104b may be housed outside of sensor module 101 in direct contact with the skin.

Device 100 may comprise one or more sensors 104. Each sensor 104 may be configured to capture, detect, and/or gather raw data characterizing a physiological or environmental parameter. Sensors 104 may include, but are not limited to at least one of: a temperature sensor, a presence sensor, a global positioning sensor, an accelerometer sensor, a gyroscope sensor, a magnetic sensor, a distance sensor, a pressure sensor, a light sensor, a biometric sensor, a blood pressure sensor, a blood glucose sensor, an oximeter sensor, a breath sensor, and the like.

In one embodiment, one or more sensors 104 comprise a brainwave detection device. For example, in one embodiment, the brainwave detection device comprises a lightweight and portable instrument based on EEG technology, whereby one or more electrodes are placed on a localized area of the head to detect specific brainwaves of interest of the user. This serves to detect brainwave status information such as, but not limited to, sleep, attention, happiness, anger, sadness, pain, anxiety, fear and excitement, where the placement of the one or more electrodes can be adjusted to suit detection of different brainwave states. For instance, the one or more electrodes can be placed near the right ear to detect brainwave states such as joy and anger, or near the forehead to detect attention.

In one embodiment, an oximeter sensor is used to measure various blood flow characteristics including, but not limited to, the blood-oxygen saturation of hemoglobin in arterial blood, the volume of individual blood pulsations supplying the tissue, and the rate of blood pulsations corresponding to each heartbeat of a user. Measurement of these characteristics has been accomplished by use of a non-invasive sensor which scatters light through a portion of the user's tissue where blood perfuses the tissue, and photoelectrically senses the absorption of light in such tissue. The amount of light absorbed is then used to calculate the amount of blood constituent being measured. An exemplary oximeter sensor can use an emitter containing two discrete wavelengths and a detector placed about 2 mm away, for example between about 10 mm and 15 mm from the emitter. The surface can be black in order to minimize any shunting of light between sensor and user skin. In one embodiment, the oximeter sensor is placed on the ear lobe, which has the advantage of providing a fast response to central changes in oxygenation or lack thereof.

In one embodiment, one or more biological data capturing sensors may include a temperature sensor and a presence sensor. The temperature sensor may comprise a thermistor, a thermal ribbon type sensor or other type of temperature sensor.

The presence sensor may be either a resistive or capacitive electrical sensor, proximity sensor or other suitable sensor configured to detect that device 100 is no longer being worn by the user. The presence sensor may be coupled on one side of the contact layer by bonding the presence sensor through heat, chemical, or adhesive methods. The presence sensor may be also integrated below the contact layer, depending on the type presence sensor. Such integration may be accomplished by weaving the presence sensor through a fabric portion of the contact layer or embedding the presence sensor beneath a cover portion of the contact layer.

In other embodiments, the presence sensor may be configured to detect that the sensor unit is positioned proximal to the body to capture raw biological data.

In one embodiment, an accelerometer sensor is configured to track the rate of linear movements of a user along one axis, two axes, or three axes, as well as the user's orientation with respect to the constant acceleration of the gravitational field vector. In one embodiment, a global positioning sensor is configured to acquire current geographical location of the user. In one embodiment, a gyroscope sensor is configured to track the rate of rotational movement of a user around one axis, two axes, or three axes. In one embodiment, a magnetometer is configured to track the direction a user is facing.

In one embodiment, device 100 comprises a control unit 110 and memory 112 electrically connected to a communication device (for example, wireless transceiver 108), one or more sensors 104 and battery 102. In one embodiment, memory 112 may include one or more non-transitory computer-readable media.

Control unit 110 is configured to receive and interpret physiological and environmental data. Control unit 110 may compare a representative value with a preset or adjustable value stored in memory 112 to further characterize a physiological condition of the body. For example, control unit 110 may compare the body temperature of a user with a preset or adjustable threshold value stored in memory 112 to determine that the temperature of the body is relatively too high or too low. In another example, control unit 110 may detect a rapid rise in pulse when comparing a change in pulse rate versus a threshold value stored in memory 112.

Control unit 110 may detect whether device 100 has been put on a user. Control unit 110 may receive data from the presence sensor indicative of the presence of a user, for example a signal representative of capacitance or resistance generated by the presence sensor. Control unit 110 may interpret this data as indicating that a user is proximal to one or more sensors 104 and may infer that one or more sensors 104 is in a position to receive reliable raw biological data, for example that it is in operational contact.

Combining readings from one or more temperature sensors, presence sensors, global positioning sensors, accelerometer sensors, gyroscope sensors, magnetic sensors, distance sensors, pressure sensors, light sensors, biometric sensors, blood pressure sensors, blood glucose sensors, oximeter sensors, breath sensors, and the like enables device 100 to provide a number of useful data, including but not limited to a user's gait speed, head rotation angles, upper body inclination angles, stability of movement, orientation, location, temperature, pulse, blood oxygen level, blood glucose level, breathing rate, blood pressure, and the like.

Wireless transceiver 108 can be any suitable transceiver for wirelessly transmitting and receiving signals, including one or more of a Bluetooth transceiver, WiFi transceiver, near field communication transceiver, mobile transceiver (e.g., 3G, 4G, etc.), and the like. Battery 102 can be any suitable battery, such as a rechargeable battery or a replaceable battery. Embodiments comprising a rechargeable battery can further comprise one or more features to enable recharging, such as a cable port for connecting to a power source. In certain embodiments, recharging is performed wirelessly by way of one or more inductive charging coils.

As described above, control unit 110 and memory 112 are electrically connected to one or more sensors 104, wireless transceiver 108, and battery 102. Control unit 110 and memory 112 may operate in conjunction with a local or remote executable software platform, or with a hosted internet or network program or portal (secondary device). As contemplated herein, any computing device as would be understood by those skilled in the art may be used with control unit 110 and memory 112, including laptops, desktops, mobile devices, tablets, smartphones or other wireless digital/cellular phones, televisions or other thin client devices as would be understood by those skilled in the art. Control unit 110 may comprise one or more logic cores. In some embodiments, control unit 110 may comprise more than one discrete integrated circuit.

Control unit 110, in conjunction with the several components of device 100, is fully configured to send, receive, and interpret device signals as described herein. For example, control unit 110 can be configured to monitor and record signals observed by one or more sensors 104 to memory 112. Control unit 110 can be configured to record some or all of the received signals from onboard components to memory 112 and subsequently interpret the signals. Control unit 110 can also be configured to record received signals from wireless transceiver to memory 112 and subsequently interpret the signals. Signals can also be recorded to cloud storage. Control unit 110 may be configured to interpret the various signals as a series of data points and subsequently transmit the data points to a digital display. Control unit 110 may further perform automated calculations based on the various signals to output information such as velocity, acceleration, orientation, angle, location, and the like, depending on the type of signals received.

Control unit 110 may comprise alerting unit 106 that is capable of emitting one or more auditory signals, presenting one or more digital readouts, providing one or more light indicators, providing one or more tactile responses (such as vibrations), and the like. Accordingly, suitable alerting units 106 include but are not limited to: speakers, alarms, lights, LEDs, vibration motors, screens, and the like. For example, certain received signals and data outputs may indicate a user has fallen and is unable to recover, whereupon control unit 110 may present one or more signals remotely to communicate the user's status and need for assistance. In some embodiments, control unit 110 communicates received signals and data outputs in real time.

Control unit 110 may further provide a means to communicate the received signals and data outputs, such as by projecting one or more static or moving images on a secondary device such as a screen. A secondary device may also comprise an external alerting unit 106 that is also capable of emitting one or more auditory signals, presenting one or more digital readouts, providing one or more light indicators, providing one or more tactile responses (such as vibrations), and the like.

In some embodiments, device 100 is used for communication. In one embodiment, a video camera (also referred to as camera sensor) is attached to device 100. In other words, in one embodiment, the at least one or more sensors 104 comprises a camera sensor that is coupled to control unit 110 for delivering image information from the surrounding of device 100. As device 100 may contain a Wi-Fi module as explained above, a remote user can send vibration commands (tactile responses) through the network by checking the images provided by the video camera (camera sensor).

In one embodiment, device 100 is used to detect the location of the user. In one exemplary embodiment, device 100 may incorporate a cellular telephone transmitter and receiver using G3 Internet protocol, as well as a global positioning sensor and transmitter. Control unit 110 can be adapted to evaluate the position and location information provided by the global positioning sensor. The device can transmit data that represents the location of its user at any time. This can be done in real-time and can be received and displayed by a secondary device.

In one exemplary embodiment, device 100 is used in surgery setting to secure one or more surgical instruments in place, such as endotracheal/or laryngeal tubes. The correct placement and fixation of such instruments is critical in maintaining a user's wellbeing. For example, in the case of endotracheal tubes, even a properly positioned and secured tube will often displace due to mechanical activity associated with the instrumentation and user movements. Such displacement can harm the user in several ways. Positioning of the endotracheal tube is not routinely checked again by fiberoptic endoscope, unless there are clinical signs for malposition (hypoxemia, changing inspiratory and expiratory tidal volumes, etc.). In one embodiment, device 100 can be used as the anchoring place to secure the airway circuit in place.

In one embodiment, using adhesives, an instrument may be coupled to device 100. In another embodiment, an instrument can be coupled to instrument holding/alignment unit 120 of the device 100 through attachment means readily known in the art, including but not limited to adhesives, Velcro, snap fits, buttons, and zippers.

The herein presented device 100 may be applied in many daily life situations. Several other practical appliances are generally conceivable. It is to be noted that the above given examples are not restrictive.

### METHOD OF USE

In one aspect, the present invention provides a method of monitoring physiological and environmental data. For example, in one embodiment, the method comprises the use of a device described herein to monitor physiological data of a user and environmental data of the surroundings. The method comprises one or more operational states. An operational state of the device may be controlled by switching between one or more modes. For example, the device may comprise an “off” mode where power to a control unit is cut off and the control unit cannot gather or interpret biological data. The off mode may be useful in conserving battery power.

The device may comprise an “idle” mode where the device is active and can send raw biological data to the control unit for interpretation. In some embodiments, the device may be activated by a wireless or wired signal sent to the control unit at the point of manufacturing, or the control unit may be pre-configured to be in the idle mode without requiring a wireless signal to activate the device. In other embodiments, the user may toggle the device between the “off” mode and the “idle” mode through mechanical switches, wireless signals transmitted to the control unit, or other methods commonly known or apparent to persons of ordinary skill in the art.

The device may comprise an “active” mode, where the device is gathering raw biological data, and the control unit is receiving such data for interpretation. The active mode may be triggered by a positive signal from the presence sensor that the sensors are in position to take reliable raw biological data.

In order to start monitoring biological data, the user may put on the device on the head, which may position a sensor to take biological data, for example in operational contact with the body. The device may be preset in the idle mode, allowing for detection of the presence of a user of the device and transition to the active more, where physiological and environmental data is monitored.

The device may be worn on the head so that the presence sensor detects that a sensor module is in position relative to the body to take reliable raw data, for example that a sensor is in operational contact with the body.

A control unit may send a signal to one or both of the on-board alerting unit and the external alerting unit located on a secondary device that generates conveys information to the user or other authorized person near the user. In operation, the control unit, via the transceiver and antenna, sends a wireless signal to the secondary device to generate the alert. In operation, the secondary device may log the event in its internal memory for recovery at a later time. The information conveyed may be the alert, such as an audible beep or other type of signaling event (e.g. a message on a display of the secondary device) to the user of the secondary device.

In operation, the control unit may determine whether the on-board alerting unit is available to generate the alert. The determination in operation may depend on whether the device includes an on-board alerting unit, or whether the user has disabled the on-board alerting unit, or whether the user has configured the control unit to send a signal to both the secondary device and the on-board alerting unit. The control unit may determine that the on-board alerting unit is available, when the alerting unit is in an “on” state and is selected for generating alerts.

In some embodiments, the control unit generates a signal to both the secondary device and the on-board alerting unit, based on determinations made in operations. In operation, the secondary device receives the command to generate the alert at the external alerting unit. In operation, the control unit may not send a signal to the on-board alerting unit based on the determination made in operation. For example, the control unit may be configured to command only the external alerting unit to generate the alert.

In one exemplary embodiment, in the active mode, the control unit may receive biometric data gathered from a user by one or more sensors. The control unit may record or write the biometric data to memory. The control unit may display the biometric data on a digital readout. The control unit may also compare the biometric data against a predetermined acceptable range stored on memory. If the received biometric data is outside of a predetermined acceptable range, the control unit may signal an alerting unit to generate an alert.

In one embodiment, one or more preset or adjustable threshold values may be stored in the memory. In other embodiments, the secondary device may be used to communicate with control unit and adjust the threshold values stored in memory.

In one embodiment, the control unit may interpret the biological data received from the sensors. In some embodiments, the control unit may make one or more comparisons of the body temperature computed with the threshold value stored in memory. It will be apparent to persons of ordinary skill in the art that the interpretation of raw biological data in may comprise other analysis of the biological data that will characterize a physiological condition of the body.

For example, the control unit may receive a temperature reading from a temperature sensor and generate a signal to indicate that the measured body temperature has not reached a threshold value. In other embodiments, the control unit may issue a command to the alerting unit to generate an alert indicating that the body temperature has not reached a threshold value within a certain time interval. Once the body temperature of the user reaches a threshold value established within the logic of the control unit, the control unit may deactivate the alerting unit.

In some embodiments, there may be multiple threshold values stored in memory. These threshold values may trigger different alerts based on the information that the user is intended to receive from the alert and based on a determination of how the raw biological data compares to the threshold value. For example, the alert generated by the on-board alerting unit may become increasingly alarming based on stepped threshold values correlating to increasingly harmful rises or falls in body temperature.

The alert provided by the alerting unit of the device to the user may not be only audible alerts; the alerts may also be vibrations (for example for the hearing impaired) generated by vibration device. Once the user receives the alert, he/she or the surgeon operating on a user will know that his/her body temperature is rising.

In one embodiment, the invention provides a method to track the location and vitals of a remote user of the device described herein. The ability to pinpoint a location while monitoring vital signs such as temperature, blood pressure, blood sugar, heart rate, and the like would be helpful for a variety of subjects, including but not limited to monitoring elderly or compromised adults who may become disoriented and wander off; keeping track of pets and people on trips; and managing dangerous situations for firefighters, park rangers, soldiers, and rescue personnel. In one exemplary embodiment, the device may also be suitable for monitoring children. In one scenario of use, a caregiver can carry a secondary device and receive wirelessly communicated alerts from a device worn by a child. In one scenario of use of the device, a caregiver can be close enough to a child wearing the device to hear audible alerts generated.

In one embodiment, the present invention provides a method of monitoring hypothermia in a subject during a surgical procedure, using the device described herein. In another embodiment, the present invention provides a method of securing an instrument, such as an endotracheal or laryngeal tubes, in place during surgery, using the device described herein

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A device for monitoring and detecting biological and environmental data, comprising:
a hat having an outer surface and an inner surface;
one or more sensors;
one or more control units operatively connected to the one or more sensors;
an alerting unit operatively connected to the one or more control units; and
at least one instrument holder attached to the outer surface.

2. The device of claim 1, wherein the alerting unit comprises one or more speakers, alarms, LEDs, vibration motors and screens.

3. The device of claim 1, further comprising a memory unit operatively connected to the one or more control unit, wherein the memory unit stores a set of predetermined acceptable biometric ranges.

4. The device of claim 1, further comprising a presence sensor operatively connected to the one or more control units, wherein one or more control units are configured to receive data from the presence sensor indicating that the device is being worn.

5. The device of claim 4, wherein one or more control units are configured to receive data from the presence sensor to detect that the sensors are positioned relative to the body.

6. The device of claim 1, wherein the one or more sensors are selected from the group consisting of: a temperature sensor, a global positioning sensor, an accelerometer sensor, a gyroscope sensor, a magnetic sensor, a distance sensor, a pressure sensor, a light sensor, a biometric sensor, a blood pressure sensor, a blood glucose sensor, an oximeter sensor, and a breath sensor.

7. The device of claim 6, wherein one or more sensors sense and measure biological and environmental data as parameters continuously or at regular intervals.

8. The device of claim 7, wherein the one or more control units are configured to receive the parameters from the one or more sensors and to signal the alerting unit once the received parameters are outside a predetermined range.

9. The device of claim 1, further comprising one or more flaps or extensions configured to cover a user's ears, forehead, eyes, or neck when worn.

10. The device of claim 1, wherein the at least one instrument holder is adapted to hold an endotracheal tube.

11. A method of monitoring biological and environmental data by wearing a device, the method comprising:
providing a device having one or more sensors, one or more control units, and an alerting unit;
wherein the one or more sensors are configured to sense and measure biological and environmental data as parameters continuously or at regular intervals;
wherein the one or more control units are operatively connected to the one or more sensors; and
wherein the alerting unit is operatively connected to the one or more control units and is configured to provide one or more alerts based on the signals received from the control unit;
such that the one or more control units are configured to signal the alerting unit once the parameters received from the one or more sensors are outside a predetermined range.

12. The method of claim 11, wherein the alerting unit comprises one or more speakers, alarms, LEDs, vibration motors, and screens.

13. The method of claim 11, wherein the device further comprises a memory unit configured to store the predetermined range retrievable by the one or more control units.

14. The method of claim 11, wherein the device further comprises a presence sensor operatively connected to the control unit, wherein the control unit is configured to receive data from the presence sensor indicating that the device is being worn.

15. The method of claim 11, wherein the one or more sensors are selected from the group consisting of: a temperature sensor, a global positioning sensor, an accelerometer sensor, a gyroscope sensor, a magnetic sensor, a distance sensor, a pressure sensor, a light sensor, a biometric sensor, a blood pressure sensor, a blood glucose sensor, an oximeter sensor, and a breath sensor.
